(19) United States
(12) Patent Application Publication
(10) Pub. No.: US 2021/0161584 A1
(43) Pub. Date: Jun. 3, 2021

(54) SIMPLIFIED METHODS FOR NON-INVASIVE VASECTOMY

(71) Applicant: Robert A. VAN WYK, St. Pete Beach, FL (US)

(72) Inventor: Robert A. VAN WYK, St. Pete Beach, FL (US)

(21) Appl. No.: 17/150,313

(22) Filed: Jan. 15, 2021

Related U.S. Application Data
(63) Continuation-in-part of application No. 16/700,393, filed on Dec. 2, 2019.
(60) Provisional application No. 62/917,325, filed on Dec. 3, 2018.
(60) Provisional application No. 62/995,188, filed on Jan. 16, 2020.

(51) Int. Cl. A61B 18/14; A61F 6/20

(52) U.S. Cl. CPC A61B 18/1445; A61B 2018/00589; A61F 6/20

(57) ABSTRACT

Conventional vasectomy techniques suffer from a number of disadvantages, including, for example, a substantial risk for the development of hematomas and swelling, a potential for spontaneous regeneration and undesired resumption of fertility, a need for a highly skilled surgical professional, as well as a long recovery period, accompanied by severe limitations on post-surgical activity. The vasectomy methods of the present invention eliminate the need for scrotal dissection and vas duct extraction and thereby overcome the disadvantages and deficiencies of the prior art, resulting in a rapid, reliable, non-invasive male sterilization procedure that may be readily, reliably and successfully performed by minimally skilled personnel around the world in a variety of medical settings.

### PRIORITY CLAIMS

The instant application is a continuation in part of U.S. patent application Ser. No. 16/700,393 filed Dec. 2, 2019, which, in turn, claims the benefit of U.S. Provisional Application Ser. No. 62/917,325 filed Dec. 3, 2018. The instant application also claims the benefit of U.S. Provisional Application Ser. No. 62/995,188 filed Jan. 16, 2020. The contents of these prior applications are hereby incorporated by reference in their entirety.

### TECHNICAL FIELD OF THE PRESENT INVENTION

The present invention relates to a simplified method for performing a non-invasive vasectomy.

### BACKGROUND OF THE PRESENT INVENTION

Vasectomy is surgical procedure that typically involves the removal of a portion of the ducts that carry sperm out of the testes (i.e., the vas deferens), thereby stopping the flow of sperm from the testicle to the prostate gland; once the vas deferens is interrupted, the sperm cannot be delivered and the man is rendered sterile. Currently used vasectomy methods, such as the No Scalpel Vasectomy (NSV), require that each vas deferens be dissected from the scrotum to allow the clinician to occlude and divide the vas duct. Therein, the vas deferens is isolated, extracted, or otherwise delivered from the scrotum via one or two openings formed by puncturing the scrotum and then expanding the opening(s). The vas sheath is then retracted from a portion of the vas duct, which is then hemi-dissected and occluded, preferably by means of mucosal cautery in which the distal end of the filament of a battery powered cautery unit is inserted into each duct lumen and energized so as to create a luminal plug of scar tissue. Alternatively, vas occlusion may involve ligation with a suture or surgical clip. In either case, after the vas is divided, a portion of the duct is optionally excised and one end is isolated in the vas sheath to create a barrier to reconnection of the duct. For example, a layer of the vassal sheath may be placed between the two severed ends of the vas in order to cover one end but not the other in a technique referred to as “fascial interpositioning”. Once both ends are sufficiently secured, the duct is then returned to the scrotum, the opening through which the vas was accessed is allowed to close and the procedure is deemed complete.

While the procedure appears simple, significant surgical skill is required, and complications may result. Most common of these is the arisal of hematomas caused by slow bleeders at the site of the duct occlusion and division. In non-elastic tissue, a small amount of bleeding is quickly stopped by the tension that develops in the tissue. However, because the scrotum is essentially an elastic balloon-like vessel, the hydrostatic pressure necessary to stop bleeding is not present. Accordingly, even the slightest amount of persistent bleeding can cause a tremendously large hematoma. In a similar manner, rough handling of the tissue can lead to significant swelling. Even the most experienced vasectomy surgeon will occasionally encounter these problems.

Other disadvantages inherent in conventional surgical vasectomy, as exemplified by the NSV, include the prolonged surgical duration, which is generally on the order of twenty minutes or more. In addition, conventional vasectomy procedures fail to adequately account for the natural tendency of the cut ends of the vas deferens to grow back together, thereby allowing the flow of sperm to the prostate and resumption of fertility. Means for avoiding this failure have been the subject of debate among those skilled in the art, the question being whether the vas deferens should be clipped, cut, cauterized, ligated, or all of the above. Finally, because sharp instruments are used, performing a vasectomy on HIV+ patients presents a risk to the surgeon.

U.S. Pat. Nos. 8,220,464 and 8,561,615, both to Pannell et al. and both incorporated herein by reference in their entirety, represent an attempt to address the afore-noted drawbacks. In particular, Pannell et al., in U.S. Pat. Nos. 8,561,615 and 8,220,464 (hereinafter referred to as “Pannell '615” and “Pannell '464”, respectively), describe devices and minimally invasive methods in which the vas duct is not dissected from the scrotum, but rather occluded percutaneously by radio frequency (RF) energy applied to the site by a bipolar coagulating device. Thereafter, a portion of the occluded duct and surrounding scrotal tissue is removed by means of an integral cutting member that serves to excise and thus permanently divide the duct.

However, as noted in co-pending U.S. patent application Ser. No. 17/700,393 filed Dec. 2, 2019 referenced above, which as published to Robert Van Wyk as US 2020/0170831 A1 on Jun. 4, 2020 (hereinafter, “the '393 application” or, alternatively, “Van Wyk '831”), the inclusion of such an integral cutting member increases the complexity of the device and the associated manufacturing process. In addition, when occluding a vas duct by the above Pannell method, the clamp that maintains the position of the duct in the fold and locates the fold in the jaws of the coagulating device must be removed before excising the tissue portion. As removal of the clamp may allow the coagulated tissue to be displaced in the jaws before or during excision of the tissue, it accordingly, it may be necessary for the clinician to exercise extreme care since displacement of the tissue may result in incomplete excision of the uncoagulated central tissue portion. Moreover, given that tissue shrinks and forms a smooth lubricious surface when coagulated, such displacement may readily occur. Thus, a goal the '393 application was to simplify the vasectomy process so as to allow those less skilled to perform the procedure, as well as to overcome existing disadvantages and deficiencies in the existing prior art.

In that vein, the '393 application teaches clamps (i.e., “excising clamps”) that serve not only to maintain the location of the duct in a fold of the scrotum, but also to excise the tissue portion when coagulation is complete according to the methods of U.S. Pat. No. 8,561,615. These excising clamps and their associated bipolar coagulating device represent a simplification over the devices of U.S. Pat. No. 8,220,464. However, although minimally invasive, the '393 application nevertheless requires surgical intervention that can prove problematic for the unskilled clinician and moreover, extend the requisite recovery period and/or impose severe limitations on post-surgical activity. Accordingly, it is a goal of the present invention to improve upon the vasectomy techniques of the '393 application by eliminating altogether any active excision step.

### SUMMARY OF THE PRESENT INVENTION

As noted above, the '393 application describes the surgical removal of a portion of the vas duct, generally via a cooperating cutting action between an excising clamp and the coagulating jaws of the electrosurgical handpiece. See, e.g., FIGS. 32B and 56 (excise duct portion 27) and FIG. 81, inset C (tissue portion 30). The instant application avoids the need for active removal. In particular, in the methods of the present invention, a vas duct is isolated within a fold of scrotal skin and clamped between the arcuate jaws of a bipolar coagulating device. Using RF energy, the portion of the fold of scrotal tissue and the vas duct positioned therein that is clamped between the jaws is fused by coagulation so as to occlude the duct in two places. Coagulation of the tissue prevents blood flow to the central uncoagulated region. Because of this, tissue in this region will necrose and slough from the body so as to divide the vas duct. Optionally, a clamp may be used to maintain the position of the vas duct in the fold of scrotal skin, the clamp being removed when coagulation is complete.

As noted above, the present invention is characterized by substantial advantages not found in conventional methods and devices. For example, by avoiding both dissection and excision, the non-invasive methods of the present invention effectively eliminate bleeding and the associated the risk for the development of massive hematomas and swelling. In addition, the present invention allows for the separation of the vas deferens in such a manner that it is virtually impossible for the ends of the vas deferens to contact each other and rejoin. Also, as compared to vasectomy methods currently available, the inventive procedure utilizes no surgical steps and thereby reduces the opportunity for complications. The inherent simplicity of the disclosed procedures and associated instruments simplifies training and allows clinicians with limited experience to master their use. Moreover, the non-invasive procedures of the present invention avoid exposure to bodily fluids, which, in turn, significantly reduces risks of transmission of blood-borne diseases, such a HIV and Hepatitis, to performing clinicians.

These and other objectives can be accomplished by the invention herein disclosed. Further objects and features of the invention will become more fully apparent when the following detailed description is read in conjunction with the accompanying figures and examples. To that end, it is to be understood that both the foregoing summary of the invention and the following detailed description are of a preferred embodiment, and not restrictive of the invention or other alternate embodiments of the invention. In particular, while the invention is described herein with reference to a number of specific embodiments, it will be appreciated that the description is illustrative of the invention and is not constructed as limiting of the invention. In addition, regarding the specific objectives recited above, it will be understood by those skilled in the art that one or more aspects of this invention can meet certain objectives, while one or more other aspects can meet certain other objectives. Each objective may not apply equally, in all its respects, to every aspect of this invention. As such, the objectives herein can be viewed in the alternative with respect to any one aspect of this invention.

### BRIEF DESCRIPTION OF THE FIGURES

Various aspects and applications of the present invention will become apparent to the skilled artisan upon consideration of the brief description of figures and the detailed description of the present invention and its preferred embodiments that follows:

FIG. 1 depicts a first step in a prior art No Scalpel Vasectomy (NSV) procedure in which a vas duct is located in a fold of scrotal tissue.

FIG. 2 depicts a subsequent step in the prior art NSV procedure in which the vas duct is isolated in a fold of scrotal tissue using a ringed clamp.

FIG. 3 depicts a subsequent step in the prior art NSV procedure in which an opening is formed in the scrotum and a portion of a vas duct in its surrounding sheath is extracted from the scrotum.

FIG. 4 depicts a subsequent step in the prior art NSV procedure in which the vas sheath is stripped back from the vas duct in preparation for occlusion.

FIG. 5 depicts a subsequent step in the prior art NSV procedure in which a first side of the hemi-dissected vas duct is coagulated using a cautery.

FIG. 6 depicts a subsequent step in the prior art NSV procedure in which a second side of the hemi-dissected vas duct is coagulated using a cautery.

FIG. 7A depicts the vas duct after subsequent removal of the portion medial to the dissections in the prior art NSV procedure, with the end of the prostate leg ligated and the suture left untrimmed.

FIG. 7B depicts the site subsequent to FIG. 7, wherein the ends of the vas enclosed in the sheath are returned to the scrotum with the leg of the ligating suture extending from the puncture in the scrotum.

FIG. 7C depicts the site subsequently to FIG. 8, with the end of the prostate leg of the vas duct secured outside of the vas sheath so as to establish fascial interposition.

FIG. 7D depicts the site at completion of occlusion of the duct via the prior art NSV procedure in which the ends of the duct returned to the scrotum.

FIG. 8 is a plan view of a bipolar electrosurgical device of a vasectomy system of the present invention.

FIG. 9 is a side elevational view of the objects of FIG. 16.

FIG. 10 is a perspective view of the objects of FIG. 16.

FIG. 11A is an expanded view of the objects of FIG. 16 at location B.

FIG. 11B is an expanded sectional view of the objects of FIG. 19A at location A-A.

FIG. 12 is an expanded view of the objects of FIG. 18 at location A.

FIG. 13 is an expanded side elevational view of the distal portion of the bipolar electrosurgical device of FIG. 16 with the device in an open, unclamped condition.

FIG. 14 is a distal perspective view of the objects of FIG. 21.

FIG. 15 is a proximal perspective view of the objects of FIG. 21.

FIG. 16 is an expanded view of the objects of FIG. 22 at location D.

FIG. 17 is an expanded view of the objects of FIG. 23 at location E.

FIG. 18 depicts a surgical system including the bipolar electrosurgical device of FIG. 8 connected to a suitable electrosurgical generator with optional foot pedal connected thereto for activation of the generator.

FIG. 19 is a perspective view of yet another alternate embodiment for an excising clamp in accordance with the present invention in a closed (clamped) condition.

FIG. 20 is an expanded view of the objects of FIG. 19 at location A.

FIG. 21 is a side elevational view of the objects of FIG. 19.

FIG. 22 is an expanded view of the objects of FIG. 2l at location C.

FIG. 23 is a plan view of the objects of FIG. 19.

FIG. 24 is an expanded view of the objects of FIG. 23 at location D.

FIG. 25 is a plan view of a portion of a scrotum with a vas duct positioned in a fold thereof.

FIG. 26 is a perspective view of the objects of FIG. 25.

FIG. 27 depicts the scrotum of FIG. 25 wherein the position of a vas duct is maintained by the clamp of FIG. 19.

FIG. 28 is an expanded view of the objects of FIG. 27 at location A.

FIG. 29 is a plan view of the scrotum and clamp of FIG. 27 wherein the jaws of the bipolar handpiece of FIG. 8 are positioned around the clamp in preparation of sealing the tissue between the jaws by coagulation.

FIG. 30 is an expanded view of the objects of FIG. 29 at location A.

FIG. 31 is a perspective view of the objects of FIG. 29.

FIG. 32 is an expanded view of the objects of FIG. 31 at location A.

FIG. 33 depicts the scrotum and clamp of FIGS. 27 and 29 after the bipolar handpiece is removed following the completion of sealing of the tissue.

FIG. 34 is an expanded view of the objects of FIG. 33 at location A.

FIG. 35 is a perspective depiction of a scrotum wherein the vas duct has been occluded according to methods of the present invention.

FIG. 36 is a plan view of the objects of FIG. 35.

FIG. 37 is an expanded view of the objects of FIG. 15 at location A.

FIG. 38 is a perspective view of an alternate embodiment clamp used in vasectomy methods of the present invention.

FIG. 39 is a side elevational view of the objects of FIG. 38.

FIG. 40 is a perspective view of the clamp of FIG. 38 in use maintaining the position of a vas duct in a scrotum.

FIG. 41 is a plan view of the objects of FIG. 40.

FIG. 42 is an expanded view of the objects of FIG. 40 at location B.

FIG. 43 is an expanded viewed of the objects of FIG. 41 at location A.

FIG. 44 is a perspective view of the objects of FIG. 40 with alternate positioning of the clamp.

FIG. 45 is a plan view of the objects of FIG. 44.

FIG. 46 is a perspective view of a tenaculum used in vasectomy methods of the present invention.

FIG. 47 is a side elevational view of the tenaculum of FIG. 46.

FIG. 48 is an expanded view of the tenaculum of FIG. 46 at location A.

FIG. 49 is an expanded view of the tenaculum of FIG. 47 at location B.

FIG. 50 is a perspective view of the distal portion of the tenaculum of FIG. 46 in use maintaining the position of a vas duct in a fold of scrotal tissue.

FIG. 51 is a plan view of the objects of FIG. 50.

FIG. 52 is a side elevational view of the objects of FIG. 50.

### DETAILED DESCRIPTION OF THE PREFERRED EMBODIMENTS

Before the present materials and methods are described, it is to be understood that this invention is not limited to the specific devices, systems, methodologies or protocols herein described, as these may vary in accordance with routine experimentation and optimization. It is also to be understood that the terminology used in the description is for the purpose of describing the particular versions or embodiments only, and is not intended to limit the scope of the present invention which will be limited only by the appended claims. Accordingly, unless otherwise defined, all technical and scientific terms used herein have the same meaning as commonly understood by one of ordinary skill in the art to which the present invention belongs. However, in case of conflict, the present specification, including definitions below, will control.

All publications mentioned herein are incorporated herein by reference in their entirety. However, nothing herein should be construed as an admission that the invention is not entitled to antedate such disclosure by virtue of prior invention.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the present invention, the following definitions apply:

The words “a”, “an” and “the” as used herein mean “at least one” unless otherwise specifically indicated. Thus, for example, reference to an “opening” is a reference to one or more openings and equivalents thereof known to those skilled in the art, and so forth.

As used herein, the noted directional terms relate to a human body in a standing position. For instance, “up” refers to the direction of the head, “down” refers to the direction of the feet. Likewise, herein, the “vertical” direction is parallel to the axis of the body and the “horizontal” direction is parallel to the floor. In a similar fashion, the term “lateral” refers to the direction extending away from the center of the body whereas “medial” refers to a direction extending toward the center of the body.

In the context of the present invention, the term “proximal” refers to that end or portion of a device or instrument which is situated closest to the body of the subject when the device is in use. Accordingly, the proximal end of an excising clamp or bipolar electrosurgical device of the present invention includes the handle portions.

In the context of the present invention, the term “distal” refers to that end or portion of a device or instrument that is situated farthest away from the body of the subject when the device is in use. Accordingly, the distal end of an excising clamp of the present invention includes the jaw components.

In the context of the present invention, the term “arcuate” is used herein to describe shapes forming or resembling an arch. It is used interchangeably with its synonym, arciform.

In the context of present invention reference invention, the terms “coagulated” or “cauterized” are interchangably used to describe a treated area of tissue. As used herein, coagulated or cauterized tissue is tissue that through the application of RF energy and pressure has been dessicated and fused to eliminate the flow of blood or other fluids.

In the context of the present invention, the term “convex” refers to a surface or boundary that curves outward, as the exterior of a sphere. Conversely, the term “concave” refers to a surface or boundary that curves inward, as to the inner surface of a sphere, or is hollowed or rounded inward like the inside of a bowl. Herein, the area of unclamped scrotal tissue defined by the U-shaped jaws of the bipolar coagulating device and the arcuate area of clamped scrotal tissue contained therein is referred to as convex in shape.

In the context of the present invention, the terms “vas”, “duct”, “vas duct” are used interchangeably and refer to the vas deferens, and may include the vas sheath as well. The terms “pure duct” or “pure vas” refer to the duct only.

Clamping devices in vasectomy methods of the present invention are used solely to maintain the position of a vas duct in a fold of scrotal skin for subsequent occlusion of the duct. Because a clamping device may contact the jaws of a bipolar handpiece during use, in order to prevent shorting of the bipolar device these clamps are formed of a dielectric material, typically a polymer or ceramic, or are formed of a metallic material and are covered with a dielectric coating. Indeed, clamps having a wide variety of configurations may be used including standard metal ring forceps and tenaculums to which a non-conductive coating has been applied.

As noted above, the present invention is characterized by substantial advantages not found in conventional methods and devices. For example, by avoiding direct dissection and resulting bleeding, the present invention is able to eliminate the risk for development of massive hematomas and swelling. In addition, the present invention allows for the separation of the vas deferens in such a manner that it is virtually impossible for the ends of the vas deferens to contact each other and rejoin. Also, the vasectomy procedure of the present invention requires fewer steps than other current vasectomy techniques, thereby reducing opportunities for complications and medical errors. Furthermore, the inherent simplicity of the disclosed procedure and associated instruments simplifies training and allows clinicians with limited experience to master their use. Moreover, the procedures of the present invention avoid exposure to bodily fluids, which, in turn, minimizes risks of transmission of blood-borne diseases, such a HIV and Hepatitis, to performing clinicians.

Although methods and materials similar or equivalent to those described herein can be used in the practice or testing of embodiments of the present invention, the preferred methods, devices, and materials are depicted in the accompanying figures and described hereinafter. However, the embodiments described herein are merely intended to illustrate the principles of the invention. Those skilled in the art will recognize that variations and modifications may be made to the embodiments without changing the principles of the invention herein disclosed. Accordingly, the accompanying figures, described in detail below that depict aspects of the invention are in no way intended to limit the scope of the present invention.

### Examples

The most common method of vasectomy currently practiced is the “No-Scalpel Vasectomy” (NSV), a procedure in which the vas deferens is delivered from the scrotum via one or two openings formed by puncturing the scrotum and then expanding the opening(s). Critically, vasectomy techniques currently in use are considered to be “No Scalpel” if the openings in the scrotum are not formed by incision using a cutting device, but rather by puncturing the scrotum and then using forceps to expand the opening by stretching the tissue. As many prospective vasectomy patients find the idea of an incision on their scrotum very intimidating, the “no scalpel” name attached to the current NSV methods tends to reduce patient anxiety. In fact, in an effort to even further reduce patient anxiety, many current NSV techniques create a single opening on the mid-line of the scrotum and both vas ducts are occluded through this opening. Nevertheless, the fact remains that conventional NSV requires extraction of the vas duct from the scrotum and thus the formation of an opening of sufficient size to permit surgical access.

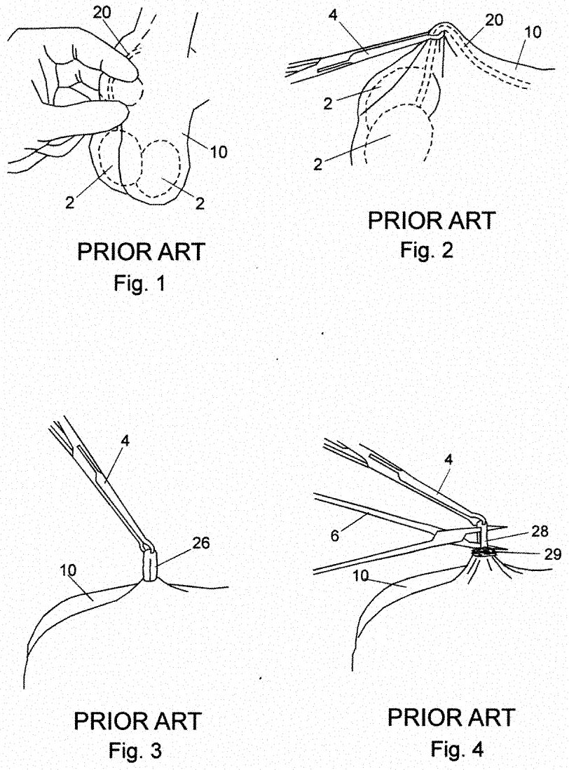

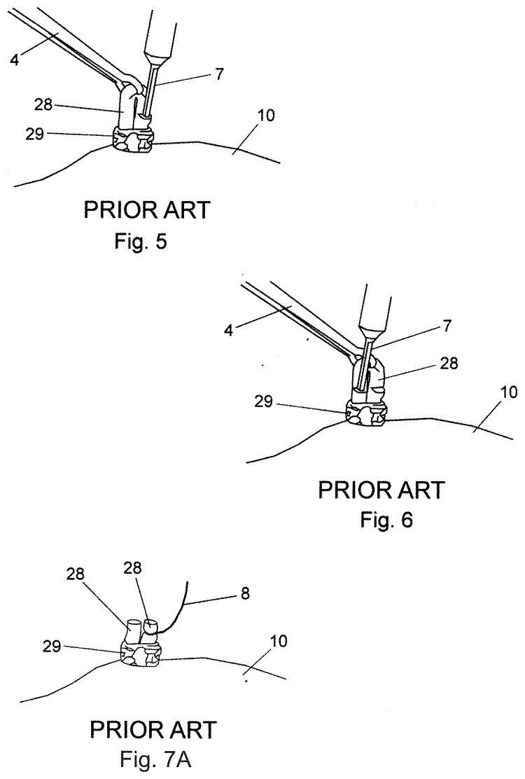

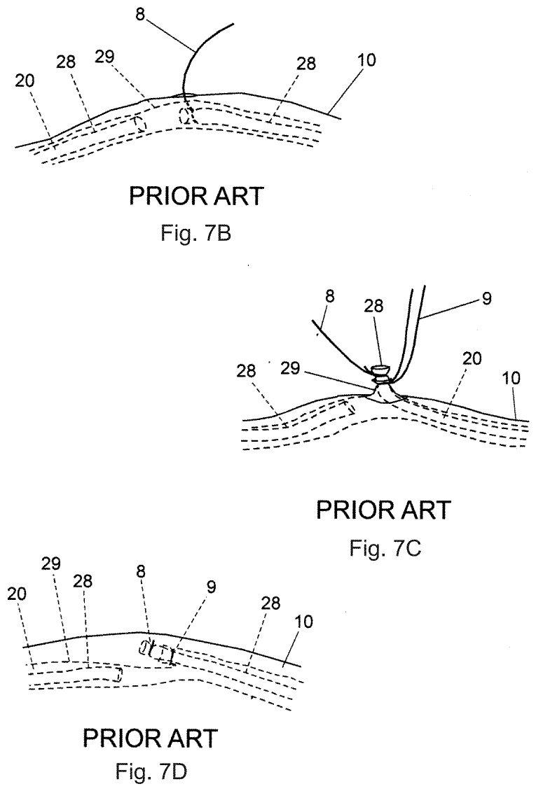

To that end, steps of a typical prior-art NSV wherein a vas duct is occluded are depicted in FIGS. 1 through 7D. In FIG. 1, a vas duct 20 is located in scrotum 10 using a standard three-finger technique. Thereafter, a local anesthetic is injected at the site. Duct 20 is then isolated in a fold of scrotum 10 using a ringed forceps 4 as shown in FIG. 2. The scrotum is then punctured using a dissecting forceps and the opening expanded sufficiently to allow the surgeon to deliver a portion 26 of vas duct 20 as depicted in FIG. 3. Dissecting forceps 6 are then used to puncture vas sheath 29 and then strip sheath 29 back to expose duct portion 28 as shown in FIG. 4. In FIGS. 5 and 6, duct portion 28 is hemi-dissected into abdominal and testicular portions, after which the distal element of an electrocautery 7 is inserted into the lumens of the respective portions and activated so as to form scar tissue in the lumens and thereby occlude them. Thereafter, as shown in FIG. 7A, a suture 8 is applied to the abdominal leg of the separated duct portion 28. Next, vas sheath 29 with the testicular portion of separated duct 28 are drawn back into scrotum 10 with suture 8 extending through the opening in sheath 29 and the opening in scrotum 10 as shown in FIG. 7B. In FIG. 7C, suture 8 is used to draw duct 28 and sheath 29 out of scrotum 10, and to draw the abdominal side occluded end of duct 28 out of sheath 29, whereupon suture 10 is tied around a portion of sheath 28 and duct 29 as depicted in FIG. 7C. Placing suture 9 in this manner permanently places a wall of sheath 29 between the divided occluded ends of duct 28 so as to provide an additional barrier to reuniting of the divided ends. FIG. 7D depicts the site with occluded, divided vas duct 20 returned to scrotum 10 with the duct ends being separated by fascial interpositioning.

As discussed elsewhere herein and as the above-referenced figures demonstrate, a typical NSV procedure involves multiple steps and requires extensive surgical skills. Completing the procedure generally requires twenty minutes or more. If the surgeon fails to notice and address any bleeders, hematomas may result. Because the scrotum is a flexible expandable vessel, these hematomas may become massive, resulting in pain and anxiety for the patient. In all cases it is necessary for the patient to restrict activities following the procedure, frequently for a week or more.

While the methods of the present invention may also be referred to as “no scalpel”, in that no incision is made in the scrotum, the instant methods in fact transcend the “no scalpel” designation in that no opening(s) are formed in the scrotum and thus the vas is not delivered from the scrotum but rather accessed in situ. Thus, in contrast to existing NSV procedures, only the methods of the present invention may be truly characterized as “non-invasive”.

In addition, the non-invasive methods of the present invention enable a clinician to perform a vasectomy in less time and with a decreased likelihood of complications. For example, in the current NSV technique, occluding of the duct, dividing of the duct, and creating the fascial interposition are accomplished in three separate steps. However, using the methods of the present invention, these three tasks may be accomplished in a single step, Thus, as less surgical skill is required, the procedure may be performed by a non-surgeon on the medical staff, for example, a nurse, nurse practitioner, or physician's assistant.

To wit, in the vasectomy methods of the present invention, a vas duct is located in the scrotum and manipulated into a fold of tissue, preferably in a high lateral position. The position of the duct in the fold is maintained using a surgical clamp, the clamp being either positioned just distal to the duct, or aligned with the mid-portion of the duct. Thereafter the jaws of a bipolar coagulating device, such as described in Pannell '464, Pannell '615, or Van Wyk '831, the contents of which are enumerated and incorporated above, are positioned around the clamp and closed onto the tissue so as to compress an arcuate region of tissue surrounding the clamp. Radio Frequency (RF) energy from the bipolar outputs of an electrosurgical generator is applied to the jaws so as to coagulate the tissue compressed between the jaws of the handpiece. This action seals the two vas duct portions clamped between the jaws along with scrotal tissue clamped between the jaws while a small region of tissue scrotal tissue and a third duct portion between the two sealed portions remain uncoagulated. When coagulation is finished the handpiece and clamp are removed from the site to complete the procedure. The vas duct is now occluded in two locations with an uncoagulated portion between the occlusion sites. While the uncoagulated tissue is not actively excised or otherwise removed during the procedure, because the aforementioned coagulation blocks the blood supply to this tissue, it will necrose and slough off naturally over time, thereby dividing the vas duct.

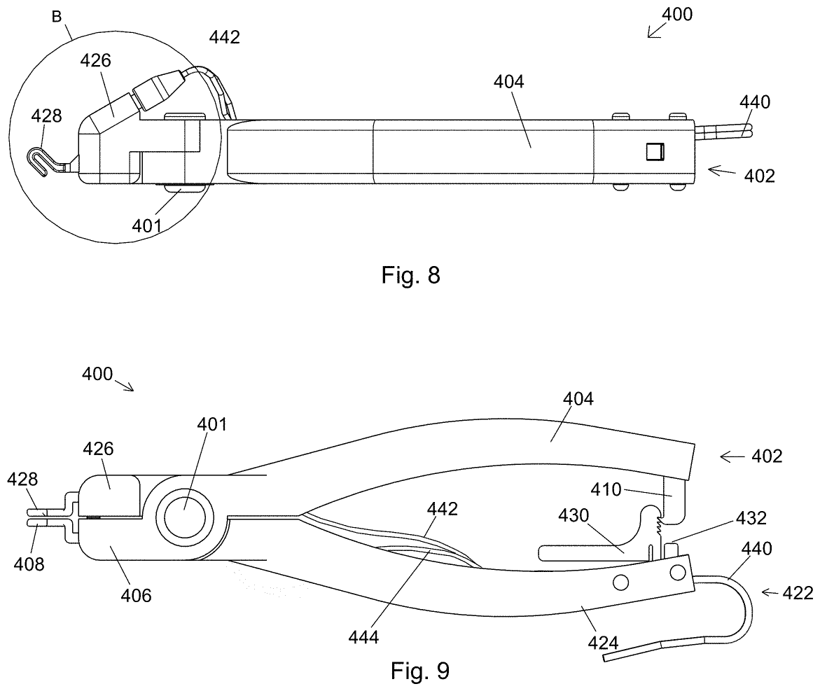

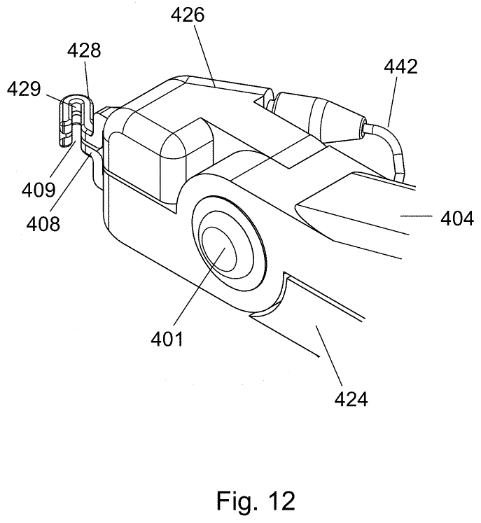

A bipolar coagulating device (handpiece) 400 suitable for use in methods of the present invention is depicted in FIGS. 8 through 12 with the jaws in a first, clamped position. Handpiece 400 is substantially similar to the equivalent electrosurgical device described in Pannell '464, Pannell '615, and Van Wyk '831 and operates by an analogous procedure. To wit, bipolar handpiece 400 has an upper handle assembly 402 with a proximal handle portion 404 and a distal portion 406 wherein is mounted lower jaw 408. Handpiece 400 has a lower handle assembly 422 with a proximal handle portion 424 and a distal portion 426 wherein is mounted upper jaw 428. Upper handle assembly 402 and lower handle assembly 422 are rotatably joined by element 401. Lower handle assembly 422 has located adjacent to its proximal end ratchet element 430 that, in cooperation with downward extending proximal portion 410 of upper handle assembly 402 maintains the clamping force of jaws 408 and 428, portion 432 of ratchet element 430 limiting the interjaw force that can be applied. Bipolar cable 440 is connected at its proximal end to the bipolar outputs of a suitable electrosurgical generator, and at its distal end, via wires 442 and 444 to upper jaw 428 and lower jaw 408 respectively such that Radio Frequency (RF) energy from the generator is conducted to jaws 408 and 428 so as to coagulate tissue clamped therebetween. In a preferred embodiment, RF energy from the electrosurgical generator is modulated according to an algorithm in the generator for maximal coagulation of tissue between the jaws.

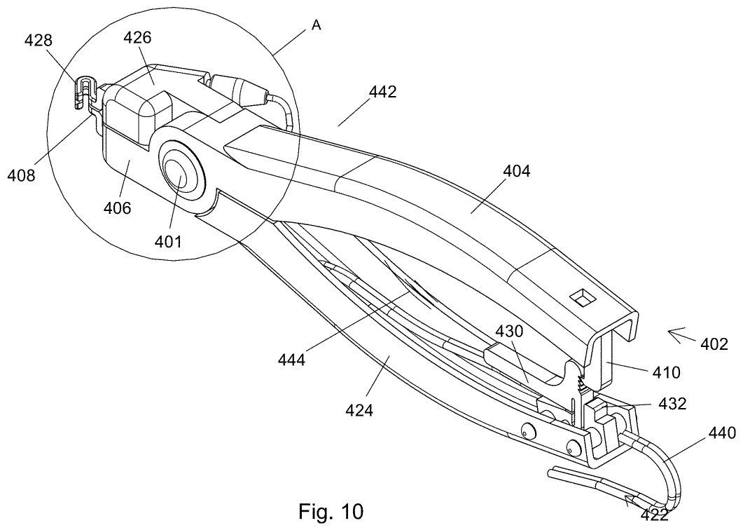

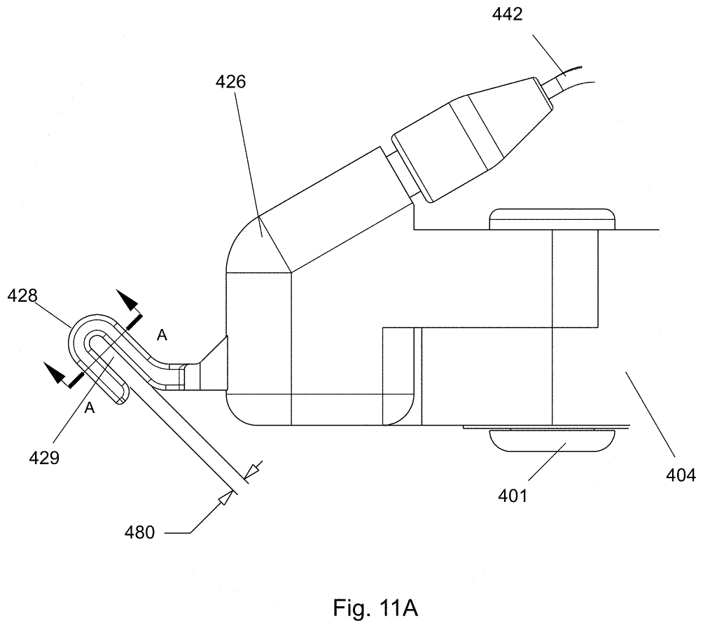

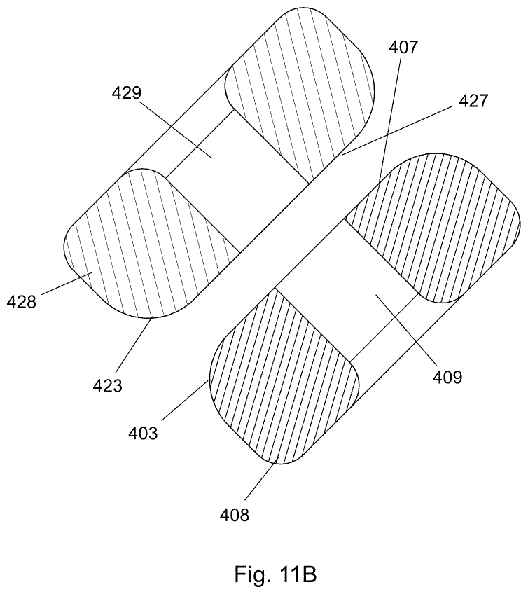

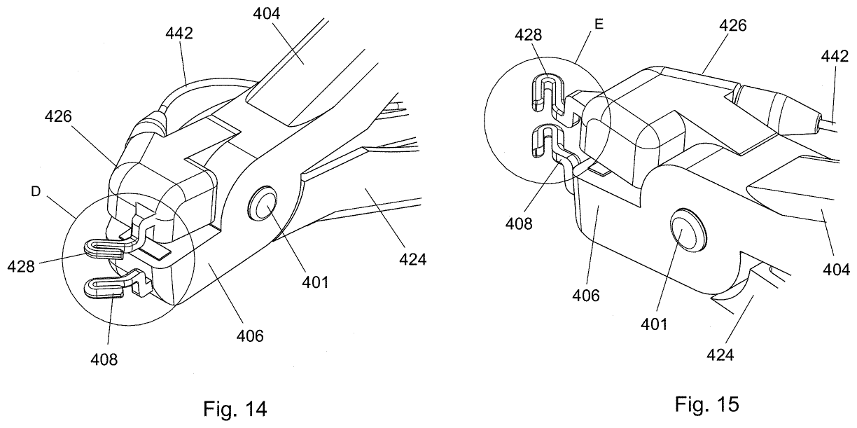

As best seen in the close-up views of FIGS. 10-12 and 14-15, upper 428 and lower 408 jaws are mirror images, each including a proximal portion that attaches to the distal end of the handpiece and a distal portion that is off-set from the longitudinal axis defined by the handpiece, preferably disposed at an angle of about 45 degrees. The angular offset affords the surgeon better visibility and access to the target surgical site. As best seen in FIG. 11A, upper jaw 428 has a “U” shape with a central slot 429 of width 480, with lower jaw 408 having a corresponding shape so that tissue may be clamped between the U-shaped jaw portions of jaws 408 and 428.

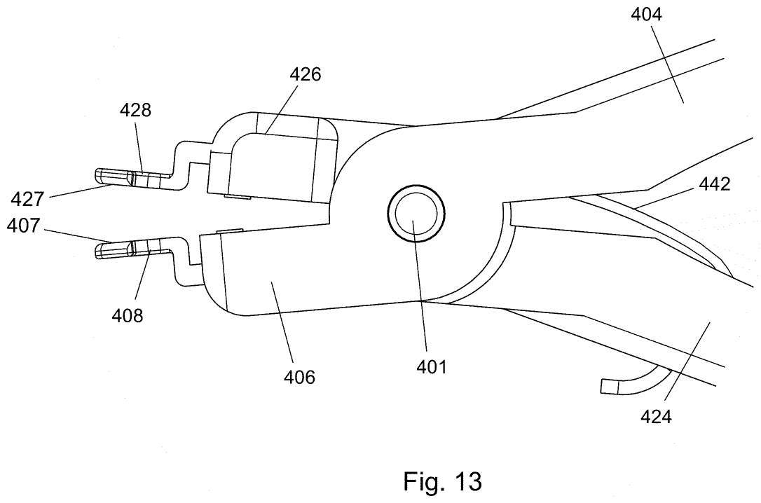

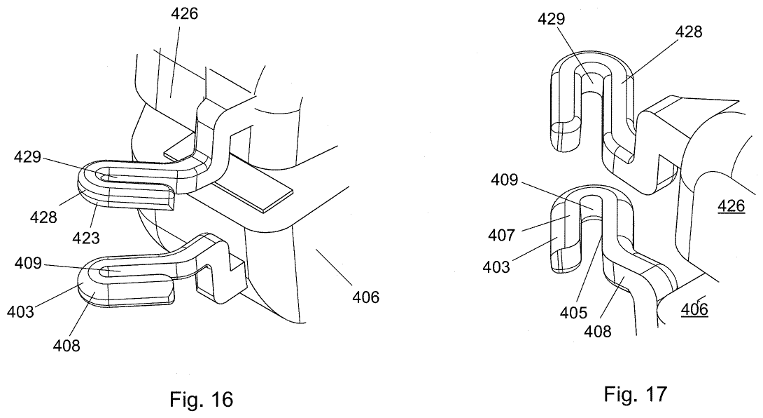

Referring now to FIG. 11B, the U-portions of jaws 408 and 428 preferably have radiused outer circumferential portions 403 and 423 respectively adjacent to their clamping surfaces to prevent cutting of tissue clamped between jaws 408 and 428. In a preferred embodiment, each offset central slot defined by each “U-shaped” distal portion is approximately 1-3 mm in width. The distal portion of handpiece 400 with handpiece 400 in its second (unclamped) position is depicted in FIGS. 13 through 17. Jaws 408 and 428 are ideally formed of a stainless steel or other suitable metallic material.

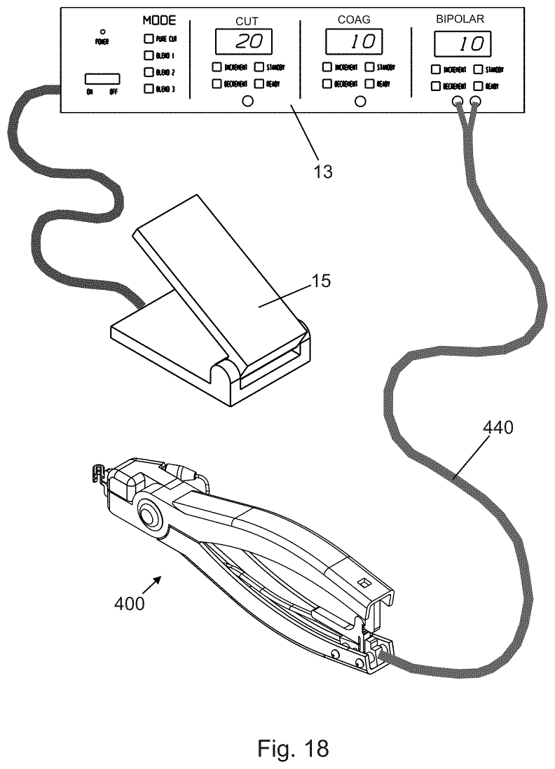

FIG. 18 depicts a bipolar coagulating device 400 connected by cable 440 to the bipolar outputs of electrosurgical generator 13 that is suitable for use in connection with the inventive methods. In the depicted preferred embodiment, generator 13 is activated by foot pedal 15. While not shown, it is understood that electrosurgical generator may be powered by alternating current, for example, via a conventional wall socket, or alternatively may be powered by direct current, for example, by means of an included rechargeable power source.

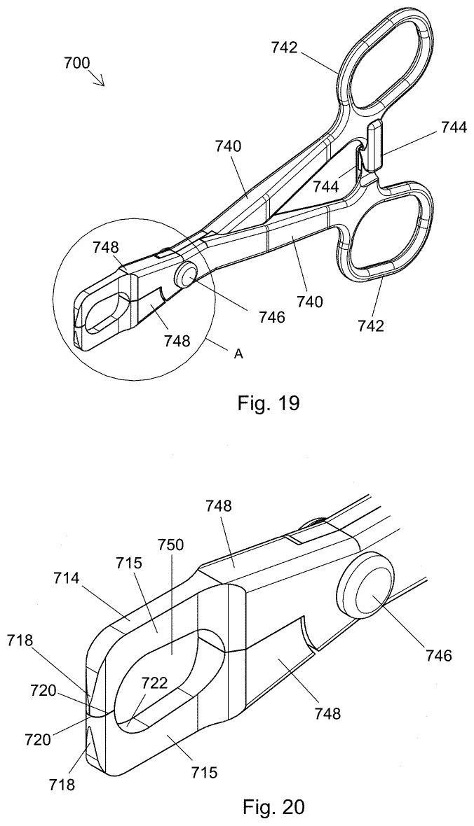

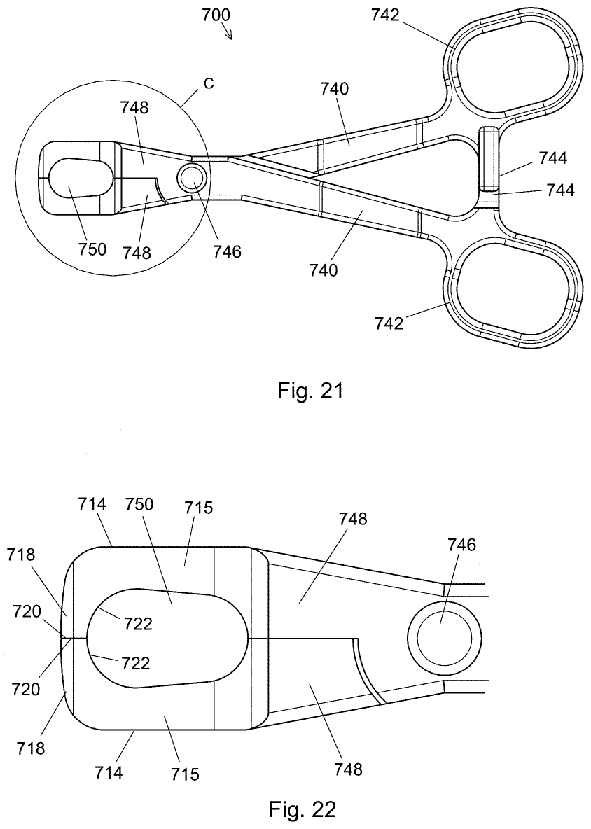

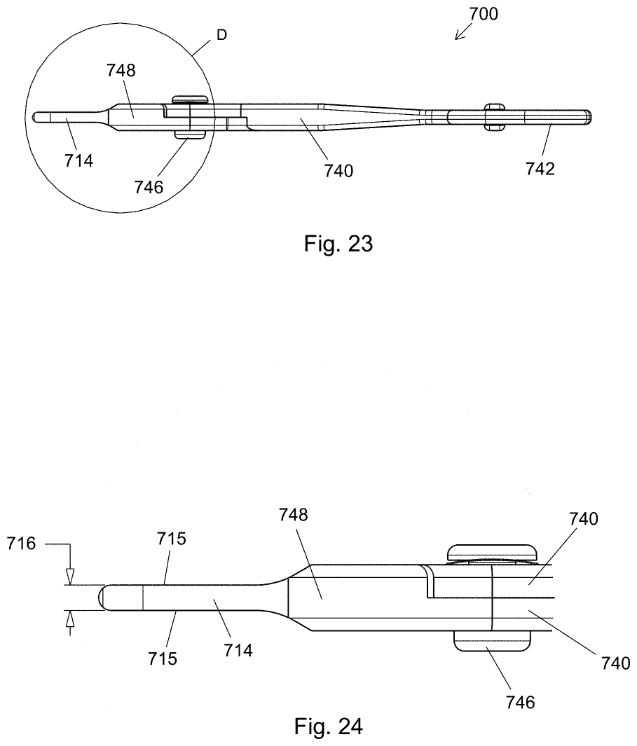

In FIGS. 19 through 24, clamp 700 is formed of elements 740 having proximal portions that form finger holes 742, and whereon are formed ratchet portions 744. Elements 740 are pivotably joined by element 746. Distal to element 746, distal portions 748 of elements 740 have a distal-most portion 714 of width 716 (FIG. 24) that is less than width 480 of slots 429 and 409 of jaws 428 and 408 respectively (see FIG. 11A). Distal-most portions 714 have at their distal ends jaw portions 718 with vertically opposed, planar jaw faces 720. Distal-most portions 714 have laterally opposed surfaces 715, and surfaces 722 that are perpendicular to surfaces 715, and that together define distal opening 750 of clamp 700. Clamp 700 may be made from a suitable dielectric material or from a metallic material with the distal portions 714 coated with a suitable dielectric coating so as to prevent shorting of bipolar handpiece 400 during use.

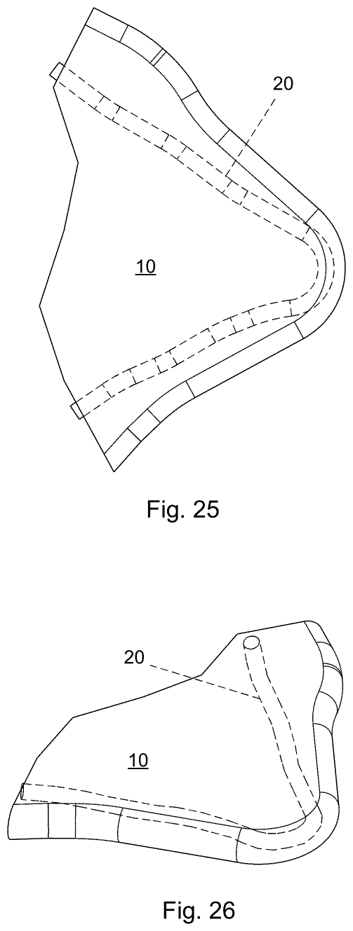

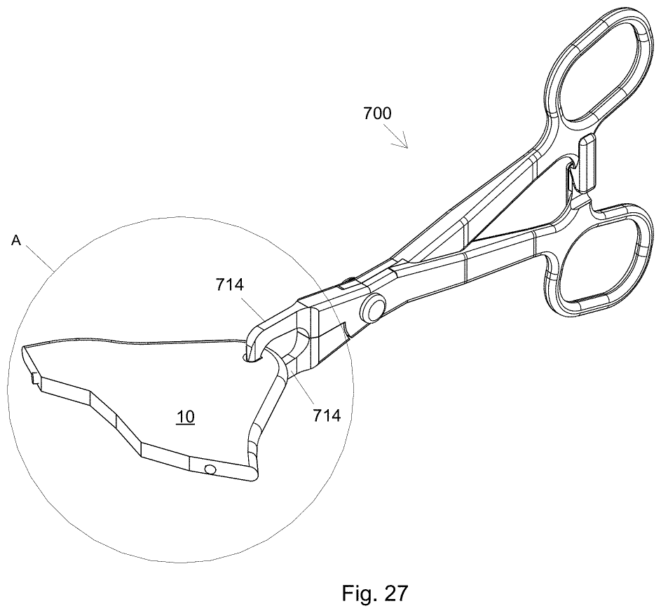

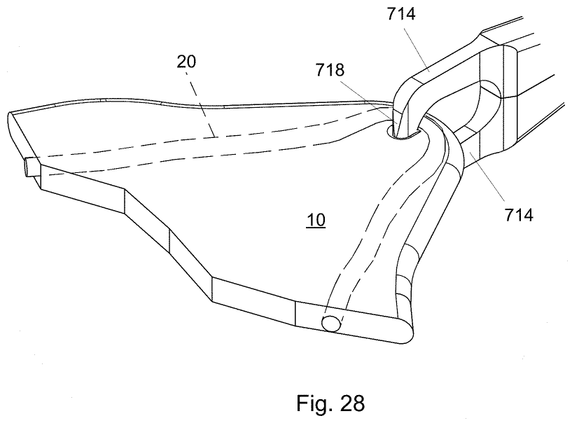

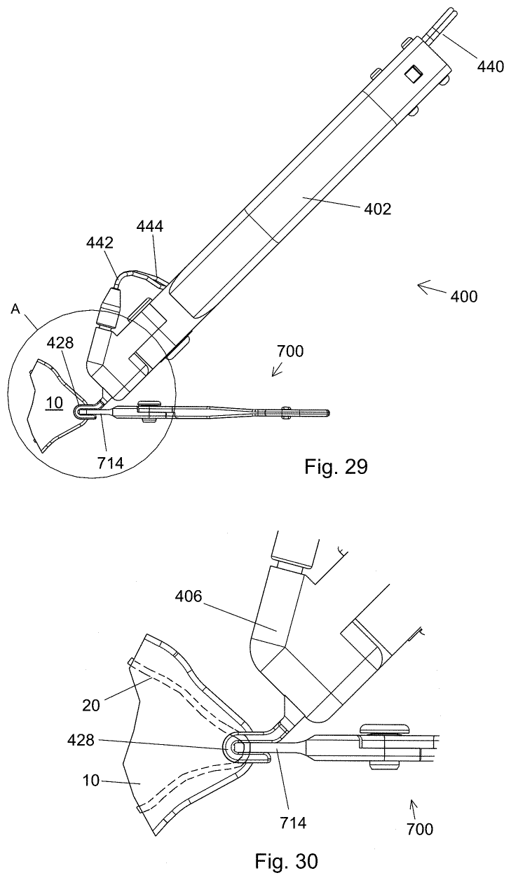

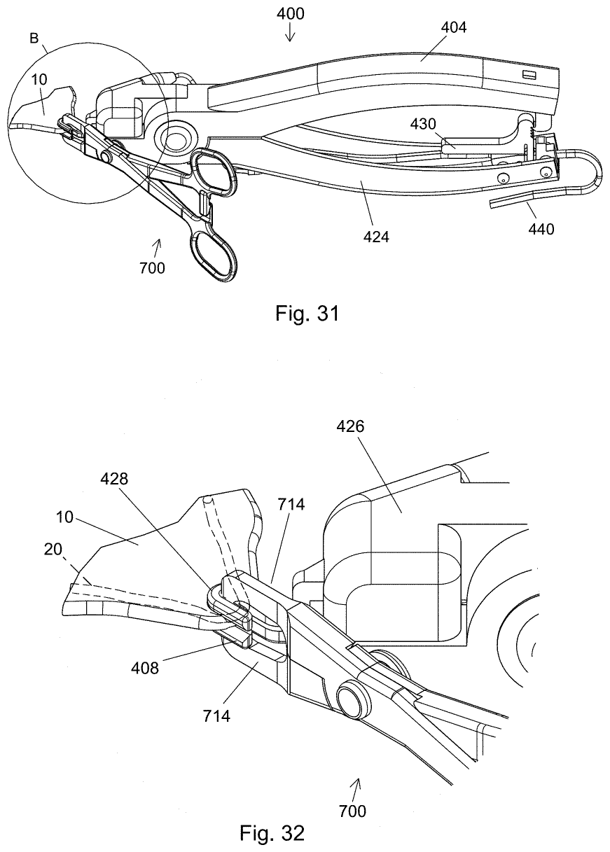

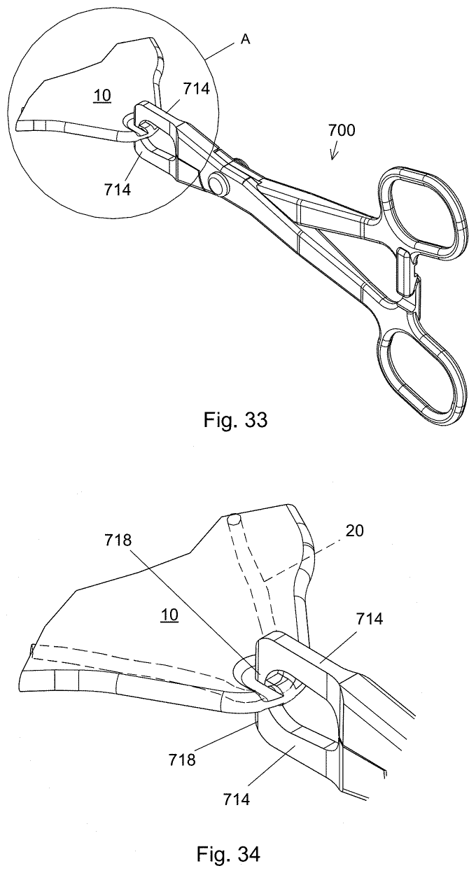

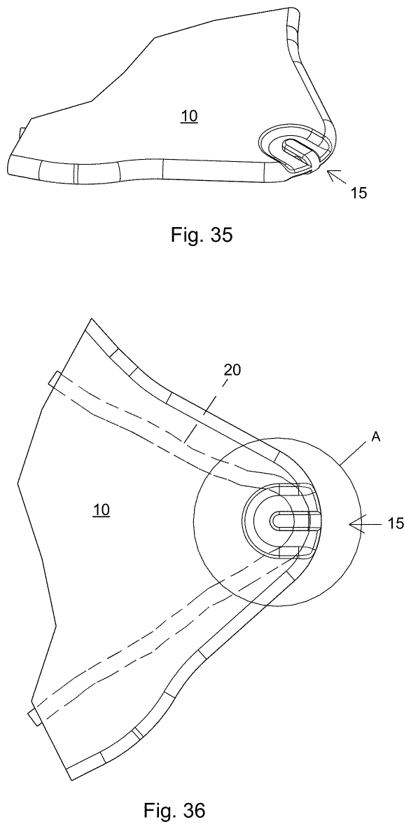

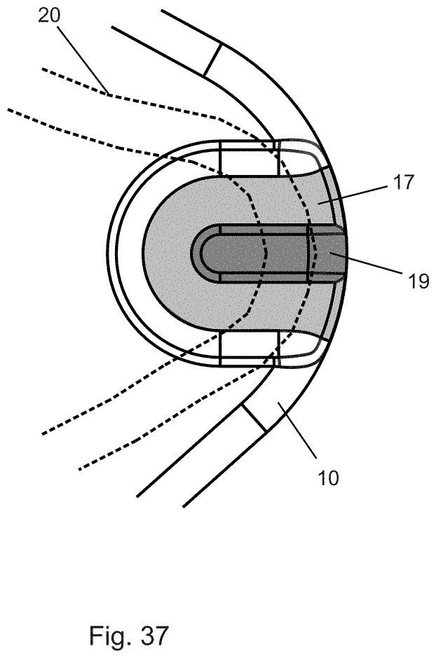

In a first step of a vasectomy procedure according to methods of the present invention, a first vas duct is isolated in a fold of scrotal skin as depicted in FIGS. 25 and 26 wherein duct 20 is located in a fold of scrotal skin 10. A local anesthesia is injected at the site. In FIGS. 27 and 28, clamp 700 is applied to the fold of scrotal skin 10 with jaws 718 medial to duct 20 so as to maintain the position of duct 20 in the fold. Thereafter, upper and lower jaws 408 and 428 of handpiece 400 are positioned around distal portions 714 of clamp 700 and handpiece 400 is closed so as to apply compressive force to the tissue between jaws 408 and 428 as shown in FIGS. 29 through 32. The clamping force may be maintained by ratchet element 430 of lower handle assembly 422. Subsequently RF energy from electrosurgical generator 13 (FIG. 18) is supplied to jaws 408 and 428 by wires 442 and 444 and cable 440 so as to coagulate portions of scrotal skin 10 and vas duct 20 that are compressed between jaws 408 and 428. When coagulation is complete, handpiece 400 is removed leaving clamp 700 in place as shown in FIGS. 33 and 34. The clamp is then removed, leaving site 15 as shown in FIGS. 35 through 37. Referring to FIG. 37, site 15 contains region 17 in which scrotum 10 and duct 20 are sealed by coagulation, and region 19 which remains uncoagulated since it was not compressed between bipolar jaws 408 and 428 of handpiece 400. Region 19 has no blood supply because it is surrounded by coagulated region 17. Because region 19 has no blood supply, it will necrose and slough off thereby dividing vas 20. Tissue adjacent to site 15 will heal and when healing is complete the gap left by the necrosed tissue will blend into the normal contour of scrotum 10.

Because the methods of the present invention require use of a clamp solely for maintaining the location of vas duct 20 in a fold of scrotal tissue 10, and to aid in positioning the jaws 408 and 428 of handpiece 400, clamps of various configurations may be used so long as they are formed of a dielectric material or coated with a dielectric material. Nevertheless, certain illustrative clamping devices are depicted and described in Van Wyk '831, the contents of which are incorporated by reference in their entirety.

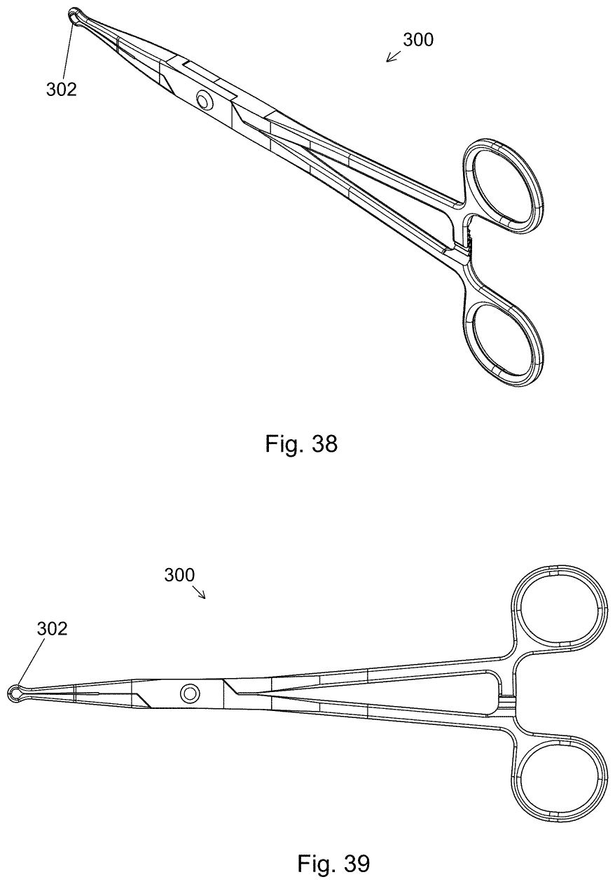

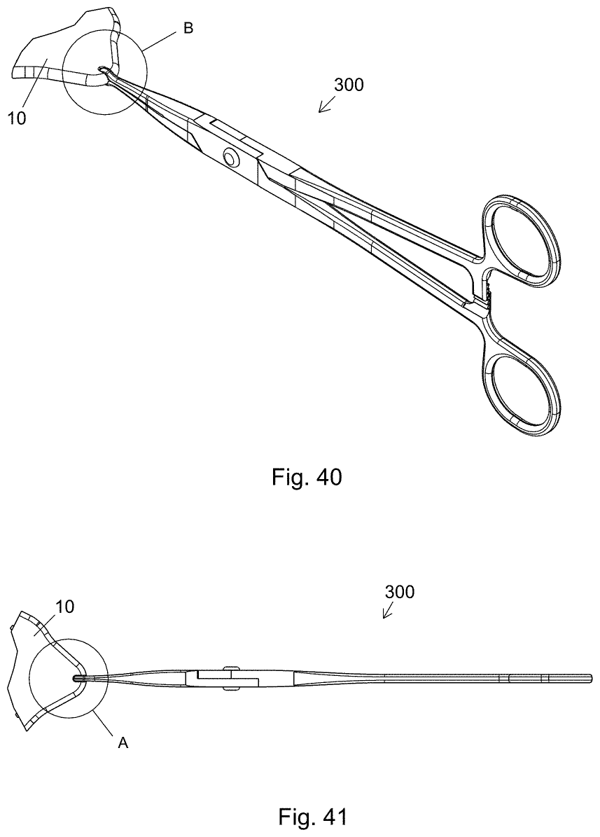

For instance, FIGS. 38 and 39 depict a ring forceps 300, an instrument commonly used in no scalpel vasectomy procedures. Ring forceps 300 is so named because of the ring shape of its distal end 302. Ring forceps 300 is used in the same manner as when doing a no scalpel vasectomy, that is to maintain the position of a vas duct in a fold of scrotal tissue. Forceps 300 is of usual metallic construction but is covered with a dielectric coating so as to not short bipolar jaws 408 and 428 of handpiece 400 when coagulating tissue. In a preferred embodiment, the device 300 is coated with parylene. Device 300 is coated in its entirety. Other embodiments are only partially coated, the coating covering the distal portion that is in proximity to jaws 408 and 428 during use. Other dielectric coatings including, for example, PTFE, may be used.

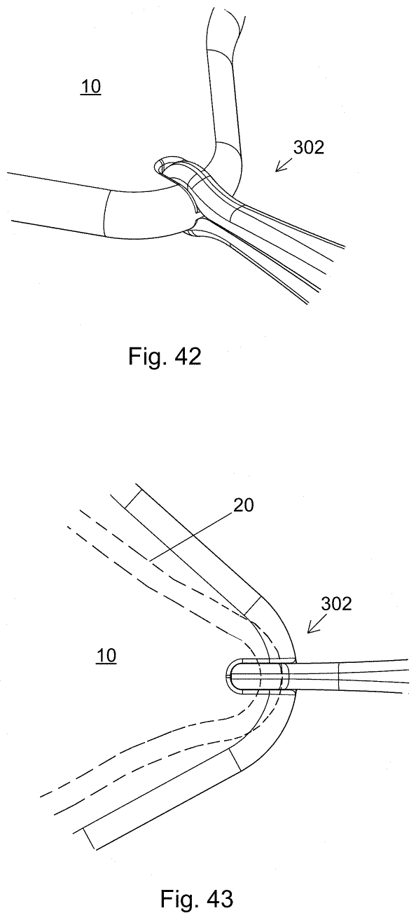

Referring now to FIGS. 42 and 43, duct 20 and surrounding tissue of scrotum 10 are maintained in position by capture within the aperture formed at distal end 302 of ring forceps 300. Jaws 408 and 428 of handpiece 400 are positioned about distal end 302 of ring forceps 300 in the manner previously herein described. Occlusion of the vas duct and sealing of the surrounding tissue proceeds in the same manner after which handpiece 400 and ring forceps are removed from the site.

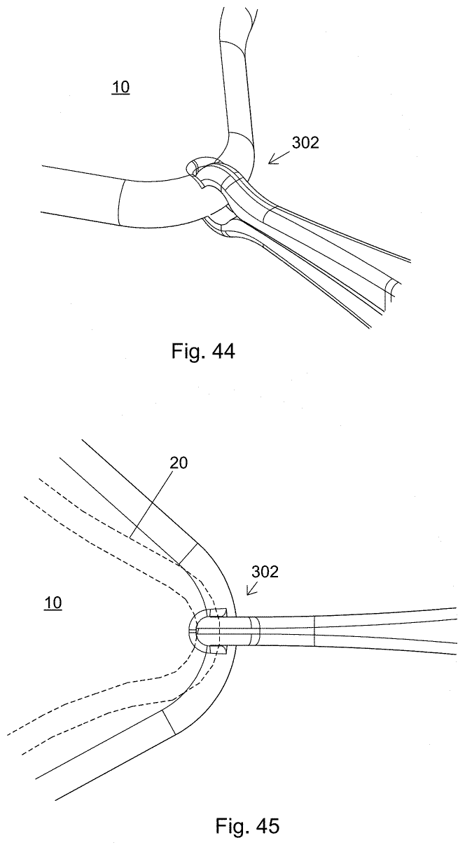

Referring again to FIG. 37, the distance to which regions 17 and 19 of site 15 extend medially into the scrotum is determined by the location of jaws of 408 and 428 of handpiece 400 during coagulation of the tissue. This, in turn, is determined by the position of the clamp around which the jaws 408 and 428 are placed. In the examples previously herein described, the clamp is placed medially adjacent to duct 20 so as to maintain the duct position. Jaws 408 and 428 are positioned medially adjacent to the clamp. It is desirable to minimize the medial extent of regions 17 and 19. This may be accomplished by modifying the location of the clamp that is maintaining the location of duct 20. For instance, referring now to FIGS. 44 and 45, distal end 302 of ring forceps 300 may be placed at the mid-line of duct 20 so as to compress duct 20 and the adjacent tissue of scrotum 10 between the clamping faces of distal end 302 of ring forceps 300. Because the position of jaws 208 and 228 is determined by the position of distal end 302, the medial extent of portions 17 and 19 of site 15 is reduced.

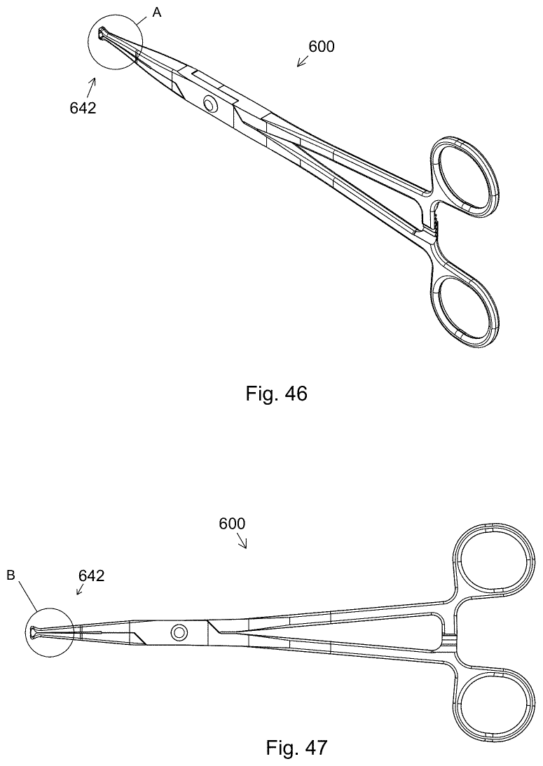

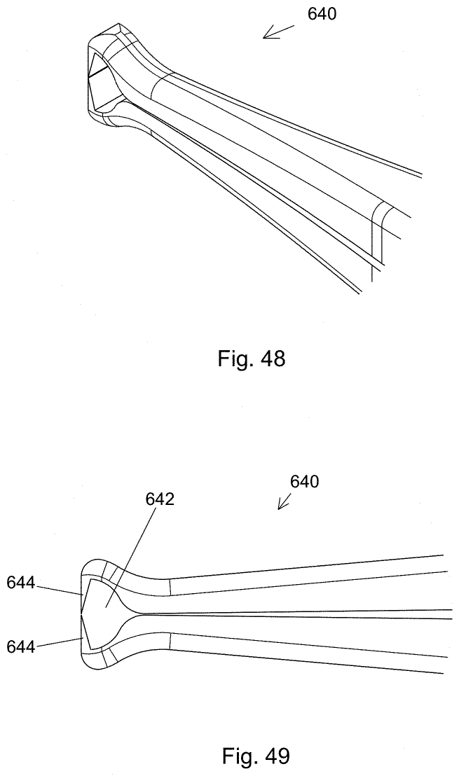

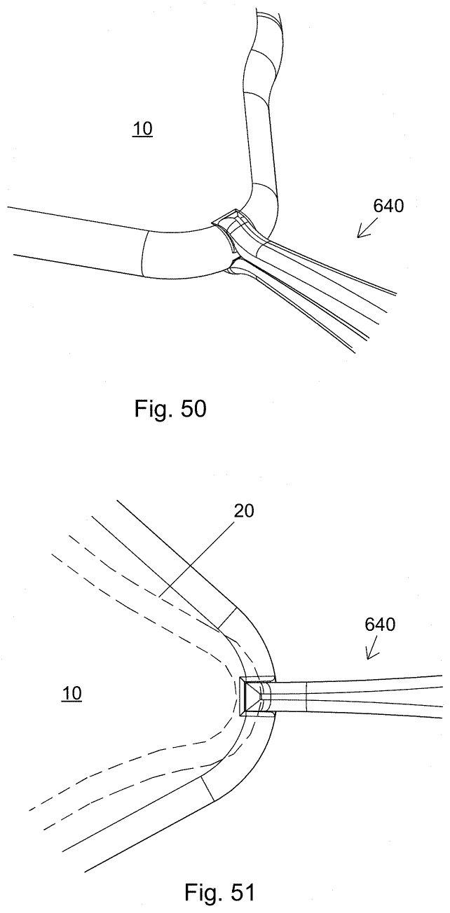

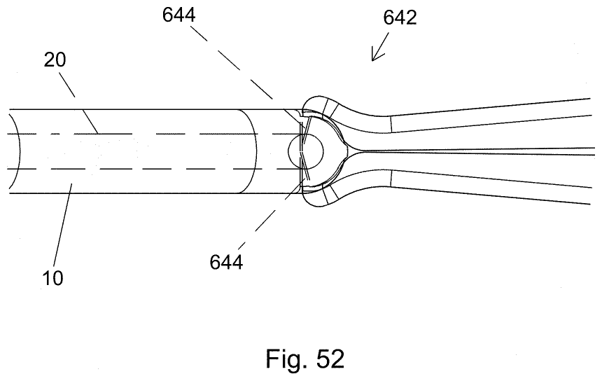

Further reduction of coagulated region 17 and region 19 that will be necrosed during healing may be realized by the use of an alternate instrument for maintaining the position of duct 20. Tenaculum 600, shown in FIGS. 46 through 49 has sharpened distal portions 644 that are configured to pierce tissue so that the tissue position may be controlled by tenaculum 600. Aperture 642 formed by distal portions 644 is configured in a manner to make the distal portion 640 depth limiting. That is, when tissue is grasped by tenaculum 600, the distance at which sharpened portions 644 intersect and penetrate tissue is limited by aperture 642. Referring now to FIGS. 50 through 52, tenaculum 600 maintains the position of duct 20 in a fold of tissue of scrotum 10, sharpened portions 644 penetrate scrotum 10 and duct 20 in the mid portion of duct 20. The positioning of jaws 408 and 428 when the location of duct 20 is maintained by tenaculum 600 is much less medial than when using clamp 700 or ring clamp 300. The medial extent of portions 17 and 19 of site 15 is commensurately reduced.

### INDUSTRIAL APPLICABILITY

As noted previously herein, by eliminating the steps of scrotal dissection and vas duct extraction, the vasectomy methods of the present invention overcome disadvantages and deficiencies of conventional vasectomy methods, providing a rapid, reliable, non-invasive male sterilization procedure that significantly reduces or eliminates negative side effects, including swelling and spontaneous regeneration, and minimizes recovery time and recovery restrictions. The methods of the present invention further avoid exposure to patient bodily fluids, thereby minimizing the potential for transmission of blood-borne diseases such as HIV and Hepatitis.

Due to the complications associated with traditional vasectomies but eliminated by the techniques and devices herein disclosed, successful procedures have, in the past, required the utilization of skilled experienced surgeons. However, the vasectomy method of the instant invention minimizes the number of steps and duration of the procedure, thereby allowing the procedure to be quickly completed by clinicians with minimal training. Moreover, given its simplicity, less skilled heath care workers can master the procedure in a relatively short period of time. This will extend the feasibility of male sterilization to areas of the world where doctors, more particularly skilled surgeons, are in short supply. For example, the method of the instant invention may be advantageously used for population control in developing countries.

While the invention has been described in detail and with reference to specific embodiments thereof, it is to be understood that the foregoing description is exemplary and explanatory in nature and is intended to illustrate the invention and its preferred embodiments. Through routine experimentation, one skilled in the art will readily recognize that various changes and modifications can be made therein without departing from the spirit and scope of the invention.

Other advantages and features will become apparent from the claims filed hereafter, with the scope of such claims to be determined by their reasonable equivalents, as would be understood by those skilled in the art. Thus, the invention is intended to be defined not by the above description, but by the following claims and their equivalents.

What is claimed:

1. A method for performing a non-invasive vasectomy comprising the steps of:
a. locating a vas duct within a scrotum;
b. temporarily isolating a section of vas tissue that includes a length of a vas duct and vas sheath in a tissue-clamping distal portion of surgical clamp;
c. providing a coagulating bipolar device having a proximal handle portion that defines a longitudinal axis of the device and a distal clamping portion characterized by a pair of opposingly-faced, upper and lower arcuate coagulating jaws that are (i) movable between open and closed positions, and (ii) provided with inner edges that engage to define a curved interior perimeter having a lateral opening that permits said upper and lower arcuate jaws to be positioned around said distal portion of said surgical clamp that retains said isolated vas tissue section that includes said length of vas duct and vas sheath;
d. tightly closing said arcuate jaws about the distal portion of said surgical clamp, thereby defining (i) an arcuate area of clamped vas tissue disposed between said closed arcuate jaws and (ii) a convex area of unclamped vas tissue containing said isolated length of vas duct retained with said curved interior perimeter;
e. activating said bipolar coagulating device so as to coagulate said arcuate area of clamped vas tissue;
f. removing said upper and lower clamping jaws from the isolated vas tissue; and
g. removing said surgical clamp,
wherein said convex area of previously clamped vas tissue containing said isolated length of vas duct remains attached to the scrotum but subsequently necroses and sloughs from the body, thereby resulting in permanent separation of the vas duct.

2. The method according to claim 1, wherein said method excludes the step of dissecting said vas tissue from the scrotum.

3. The method according to claim 1, wherein the method is performed in situ, without removing said vas tissue from the scrotum.

4. The method according to claim 1, wherein said locating step (a) further includes the step of manipulating said vas duct into a fold of scrotal tissue in a high lateral position.

5. The method according to claim 4, wherein said surgical clamp is placed medially adjacent to the vas duct.

6. The method according to claim 5, wherein said surgical clamp is a ring forceps.

7. The method according to claim 6, wherein a distal end of said ring forceps is placed at a mid-line of said vas duct so as to compress both said duct and adjacent scrotal tissue between opposed clamping faces of said ring forceps.

8. The method according to claim 5, wherein said surgical clamp is a tenaculum.

9. The method according to claim 8, wherein said tenaculum maintains the position of said vas duct in a fold of scrotal tissue, wherein sharpened portions of said tenaculum penetrate both the vas duct and the scrotal tissue in a mid portion of said vas duct.

10. The method according to claim 1, wherein pair of opposingly-faced, upper and lower arcuate coagulating jaws are angularly offset from the longitudinal axis of the device.
